(19) United States
(12) Patent Application Publication
(10) Pub. No.: US 2013/0177989 A1
(43) Pub. Date: Jul. 11, 2013

(54) Enhanced E. coli for the production of fatty acids and method of producing the same

(71) Applicants: Abel Baerga-Ortiz, San Juan, PR (US); Delise Oyola-Robles, San Juan, PR (US)

(72) Inventors: Abel Baerga-Ortiz, San Juan, PR (US); Delise Oyola-Robles, San Juan, PR (US)

(21) Appl. No.: 13/344,062

(22) Filed: Jan. 5, 2012

(51) Int. Cl. C12N 1/21; C12N 15/70

(52) U.S. Cl. 435/471; 435/252.33

(57) ABSTRACT

The invention analyzed a protein sequence using the Udwary-Merski algorithm and identified a tetradomain fragment (DH1-DH2-UMA) which consists of two predicted DH-like domains and two pseudodomains N-terminal to them. This arrangement of domains and pseudodomains is fundamentally the opposite of what is typically observed in the DH cassettes of actinobacterial polyketide synthases or mammalian fatty acid synthases, both of which feature C-terminal pseudodomains. The invention modified E coli by over expressing H1-DH2-UMA in E coli resulting in an increase in the overall production of all the fatty acids normally present in the E coli fatty acid profile.

### GOVERNMENT INTEREST

The claimed invention was made with U.S. Government support under grant number CHE-0953254 awarded by the US National Science Foundation (NSF). The government has certain rights in this invention.

### BACKGROUND OF THE INVENTION

Long-chain polyunsaturated fatty acids (PUFAs) have been implicated in human brain development as well as in the maintenance of cardiovascular health. Although animals have the enzymes necessary to form long-chain PUFAs through the elongation of plant-derived PUFAs, this oxygen-dependent process is not efficient. An efficient pathway for the biosynthesis of PUFAs in deep-sea bacteria utilizes a polyketide synthase-like (PKS-like) multienzyme complex. A total of five genes from this pathway have been found to be sufficient for the production of polyunsaturated fatty acids in an otherwise non-producing Escherichia coli. These genes are pfaA, pfaB, pfaC, pfaD, encoding PUFA synthases containing enzyme domains for acyl tranferases (AT), keto-acyl synthase (KS), acyl carrier protein (ACP), keto-acyl reductase (KR), enoyl reductase (ER) and dehydratase (DH) activities and also pfaE, which encodes a required phosphopantetheine transferase (PPTase) essential for the activation of ACP domains through chemical modification as shown in FIG. 1. While some of the required enzymatic activities are housed in independent stand-alone proteins (pfaB, pfaD and pfaE: FIG. 1) others are assembled into multidomains (pfaA and pfaC: FIG. 1). No thioesterase activity has been observed in the PUFA synthase cluster and no dedicated thioesterase protein from the producing organism is required for heterologous production of PUFAs in E coli.

Dehydratase (DH) domains are responsible for the formation of the cis double bonds in the structure of PUFAs. They can be easily identified by their sequence similarity to FabA and FabZ, the two DH enzymes involved in fatty acid biosynthesis in E coli. FabA/Z catalyze the dehydration of 3Rhydroxyacyl-ACP via a syn elimination mechanism which has also been reported in the DH domain from the erythromycin PKS.

The structure of FabA, and more recently FabZ, revealed an obligate homodimeric arrangement in which both DH subunits contribute key residues to the active site. This distinct architectural feature has been found to extend to DH domains from the animal Fatty Acid Synthase (FAS), and more recently to the erythromycin PKS, although with the following variation on the E coli arrangement. While the E coli FabA and FabZ form homodimers of identical subunits, the DH domains from FAS and PKS systems form a heterodimeric double hotdog arrangement in which two contiguous pseudosubunits are housed within the same polypeptide and separated by a 25-residue amino acid stretch. Thus, the required dimerization of the DH domain in the context of a multienzyme complex does not necessarily involve interactions between different polypeptides, but rather within the same polypeptide.

In both the FAS and PKS DH, the protein region that is homologous to FabA is followed by a necessary C-terminal pseudodomain with no previously known function and no known sequence homologue. In the case of the FAS DH, the C-terminal pseudodomain was found to contribute to dehydratase activity in in vitro enzyme assays. The structure of the PKS DH showed that the Cterminal pseudodomain forms the other half of the double hotdog in the three-dimensional structure. In that work, the protein construct that was crystallized, and whose structure was determined, contained the pseudodomain but lacked dehydratase activity in vitro, although mutations made elsewhere did show an effect on overall polyketide production by the full-length multienzyme.

The PUFA synthase multienzyme contains two putative DH domains in tandem. They have been identified as DH domains based on their sequence similarity to FabA/Z, but their activity or specificity has not been confirmed biochemically. The tandem arrangement, while not previously observed in other biosynthetic enzyme systems, is a well-conserved feature of PUFA synthases. However, it is unknown how these tandem domains act to generate the combination of double and single C—C bonds in the final PUFA structure.

### SUMMARY OF THE INVENTION

According to an aspect of the invention, a protein fragment consisting of the two tandem putative DH domains and the two corresponding pseudodomains from the PUFA synthase was designed using the Udwary-Merski Algorithm (UMA) developed at Johns Hopkins University.

According to another aspect of the invention, the resulting tetradomain fragment showed some dehydratase activity against an acyl-CoA soluble substrate. Examination of the three dimensional models for the individual domains reveal that while two domains contain all the conserved residues expected for a functional DH domain, the other two domains contain other residues present on other hot-dog proteins.

According to still another aspect of the invention, the analysis of the tetradomain sequence anticipates an “inverted” double hotdog arrangement in which the pseudodomain is actually located N-terminal to the FabA homology domain, thus providing an alternative topological solution which suggests evolutionary convergence of the DH architecture in PUFA synthase multienzymes.

### BRIEF DESCRIPTION OF THE DRAWINGS

Further features and advantages of the invention will become apparent from the following detailed description taken in conjunction with the accompanying figures showing illustrative embodiments of the invention, in which:

FIG. 1 shows a gene cluster for the anaerobic production of eicosapentaenoic acid (EPA) in Photobacterium profundum according to the present invention.

FIG. 2 shows a scheme summarizing the construction of different protein fragments for the isolation of dehydratase activity according to the present invention.

FIG. 3 shows graphs for Dehydratase activity of DH1-DH2-UMA towards crotonyl-CoA and the specific activity of DH1-DH2-UMA toward crotonyl-NAC in the hydration reaction and towards β-hydroxybutyryl-CoA in the dehydration reaction according to the present invention.

FIG. 4 shows a saturation curve obtained by measuring the activity of DH1-DH2-UMA towards crotonyl-CoA at 235 nm according to the present invention.

FIG. 5 shows a graph illustrating the over-expression of DH1-DH2-UMA in E coli resulting in an increase in the total production of free fatty acids in liquid bacterial culture according to the present invention.

FIG. 6 illustrates a comparison of the three-dimensional models obtained for the FabA-homology regions (DH1 and DH2) and for the uncharacterized pseudodomains (DH1.′ and DH2.′) according to the present invention.

Throughout the figures, the same reference numbers and characters, unless otherwise stated, are used to denote like elements, components, portions or features of the illustrated embodiments. The subject invention will be described in detail in conjunction with the accompanying figures, in view of the illustrative embodiments.

### DETAILED DESCRIPTION OF THE INVENTION

Experimental Procedures

Cloning, Expression and Purification.

Different DH fragments were cloned from fosmid 8E1. All restriction endonucleases, polynucleotide kinase, T4 DNA ligase, and alkaline phosphatase were purchased from New England Biolabs. The primers used to make the different fragments are summarized in Table 1 below. For cloning into pGEX4T-3 vector (GE Healthcare), the amplified DNA was phosphorylated using polynucleotide kinase and cloned into pUC19 which was previously digested with SmaI and treated with alkaline phosphatase. The ligation mixture was used to transform DH10B cells and clones were selected in LB-agar containing ampicillin (100 μg/mL). Insertion of the DH fragment into pUC19 was confirmed by agarose gel electrophoresis. The resulting plasmid pUC19:DH was digested with BamHI and SmaI and the resulting excised DNA fragment was cloned into the corresponding sites in pGEX4T-3.

TABLE 1

| Dehydratase | |
|---|---|
| construct | Oligonucleotide sequence |
| DH1S | Fwd: 5′-CATGCATGGGATCCAACTTGCTAGACGCAAATATCGCA-3′ |
| | Rv: 5′-CATGCATGCCCGGGTCATGATTCTTCTTTGATCATCACG-3′ |
| DH1L | Fwd: 5′-CACCTTCTCTTACGAATGTTTCGTTGGC-3′ |
| | Rv: 5′-CATGCATGCCCGGGTCATGATTCTTCTTTGATCATCACG-3′ |
| DH2S | Fwd: 5′-CATGCATGGGATCCAACTTACTGGATAAAGAAAGCCGTT-3′ |
| | Rv: 5′-TCAGGCTTCTTCAATACAGATTGC-3′ |
| DH2L | Fwd: 5′-CATGCATGGGATCCTTCAGCTTCGAACTCAGTACCGA-3′ |
| | Rv: 5′-TCAGGCTTCTTCAATACAGATTGC-3′ |
| DH1-DH2-S | Fwd: 5′-CACCAACTTGCTAGACGCAAATATCGCA-3′ |
| | Rv: 5′-TCAGGCTTCTTCAATACAGATTGC-3′ |
| DH1-DH2-L | Fwd: 5′-CACCTTCTCTTACGAATGTTTCGTTGGC-3′ |
| | Rv: 5′-TCAGGCTTCTTCAATACAGATTGC-3′ |
| DH1-DH2 UMA | Fwd: 5′-CACCCGCAAACCTTGTATCTGGGATTA-3′ |
| | Rv: 5′-TCAGGCTTCTTCAATACAGATTGC-3′ |

For the cloning of fragments into pET200TOPO, the amplified DNA was gel purified using the QIAquick Gel Extraction Kit and incubated with pET200TOPO (Invitrogen). The resulting clones were selected in LB-agar containing kanamycin (100 μg/mL). All resistant clones were introduced into E coli strain BL21-DE3-Codon Plus-RIL (Promega) and grown in liquid LB at 37° C. until the OD600=0.4 at which time the temperature was decreased to 22° C. until the OD600=0.6 at which time protein expression was induced with 1 mM IPTG. After 16 h, the cells were collected and resuspended in lysis buffer (50 mM Na3HPO4 pH 7.2, 150 mM NaCl, 1 mM DTT, 10% glycerol, 0.1 mg/mL lysozyme and DNAse) for 1 hr, sonicated and centrifuged at a speed of 14,000 rpm at 4° C. for 30 min in a J2-21 Beckman centrifuge in a JA17 rotor. Samples were collected for the total, supernatant and pellet to assess solubility of the protein products.

For His-tagged soluble proteins, the lysate was collected and poured through a column filled with Ni-NTA resin (Qiagen) equilibrated in 25.0 mM Tris pH 8.0, 150 mM NaCl, 10% glycerol, 1.0 mM DTT. The DH fragment was eluted with the same buffer containing 300 mM imidazole.

Eluted protein was infused into a HiLoad 16/10 Q Sepharose.™ High Performance column (GE Healthcare) operated at room temperature and equilibrated in 25 mM Tris pH 8.0, 150 mM NaCl, 1.0 mM DTT and 10% glycerol. The proteins were eluted in a 40-minute gradient 0.15 M-2 M NaCl. The fraction containing the protein was concentrated and stored at −80° C. Typical yields for all proteins were 1.0 mg of protein per liter of culture, purity ˜99% by 8% SDS-PAGE.

UMA Parameters.

The UMA program was used and UMA calculations were done as in (Udwary et al., 2002) using the sequence of pfaC from Photobacterium profundum (GenBank Accession no. AF409100.1). A multiple alignment of homologues of pfaC was performed in CLUSTALW in “.pir” format and a secondary structure prediction for pfaC was performed using the PSIPRED Server (University College London). The output for the secondary structure prediction was used to generate an “.ss” file. Finally, both the “pir.” alignment and the “ss.” secondary structure prediction were used as inputs for the “uma19.pl” application with the input parameters in Table 2 below. Results in the output file were visualized using Keleidagraph for Windows.

TABLE 2

| Parameter | Value |
|---|---|
| Homology matrix | blosum 30 |
| Gap to gap penalty | 0 |
| Gap to aa penalty | −4 |
| Component averaging (k) | 5 |
| Final averaging (gamma) | 20 |
| Sim score weight | 10 |
| Struc score weight | 1 |
| Hydro score weight | 5 |

Dehydratase Assays.

Dehydratase activity was measured in a hydration assay by using Crotonyl-CoA (Sigma) and Crotonyl-NAC as substrates. Crotonyl-NAC was synthesiszed from crotonic acid (Sigma) and N-acetylcysteamine (Sigma) using a DCC coupling strategy as describes by the prior art and purified by flash column chromatography on silica gel using 1:1 ethyl acetate: ethyl ether. For the dehydration assay β-hydroxybutyryl-CoA (Sigma) was used as the substrate. Enzymatic reactions were followed spectrophotometrically by monitoring the absorbance at 260 nm in a 96-well plate format on a Spectramax 190 instrument (Molecular Devices). The total volume was 200 μL (25 mM Tris, 150 mM NaCl, 10% glycerol, pH 8.0, 3.20 μM DH1-DH2-UMA, and 117 μM of substrate). The values for the absorbance slope (given in mAU/min) were converted to units of pmole of product per minute by using the following equation:

μmole of product/min=[Slope/ε×b]×Volₜₒₜₐₗ (Eq 1)

in which the slope is given by the instrument in units of milliabsorbance (mAU) per minute, b is the path length measured to be 0.89 cm for a Volₜₒₜₐₗ=200 μl in our 96-well plates. The ε is the molar extinction coefficient resulting from the loss of a double bond as defined by the difference in absorbance between crotonyl-CoA and β-hydroxybutyryl-CoA at a particular wavelength. The extinction coefficient was calculated to be ε=969.9 M⁻1 cm⁻¹ for the reaction monitored at 260 nm and ε=790.7 M⁻¹ cm⁻¹ for the reaction monitored at 235 nm.

For the kinetic assays the reaction was monitored at a wavelength of 235 nm and using a range of substrate concentrations between 0 and 600 μM. The data was fit to a simple Michaelis-Menten Equation (Eq 2) using Kaleidagraph v4.03.

Vₒ=Vₘₐₓ[S]/([S]+Kₘ) (Eq 2)

Fatty Acid Profiles.

E coli BL21-DE3-CodonPlus (RIL) cells expressing DH1-DH2-UMA in the pET200Topo vector were cultured in LB media and the expression was induced as described for protein production. Protein expression was confirmed by SDS-PAGE. Cells were collected by centrifugation at 4,400 rpm, 10 min, 4° C. and freeze-dried. The fatty acid components of the cell culture were obtained as their methyl esters by the reaction of 0.05 g of dried cell pellet with 10.0 mL of methanolic HCl, refluxed for 2 hr followed by workup with hexane twice. The organic layer was dried over MgSO₄ and concentrated in vacuo. The fatty acid methyl esters were analyzed by GC-MS (at 70 eV using a Hewlett Packard 5972A MS ChemStation) equipped with a 30 m×0.25 mm special performance capillary column (HP-5MS) of polymethylsiloxane crossed-linked with 5% phenyl methylpolysiloxane. The temperature program was as follows: 130° C. for one minute, increase at a rate of 3° C./min to a 270° C., where the temperature is maintained for 30 min. Methyl heneicosanoate (Sigma) was used as an internal standard for quantification of fatty acid methyl esters.

Results

Design and Expression of Putative DH Domains from the PUFA Synthase.

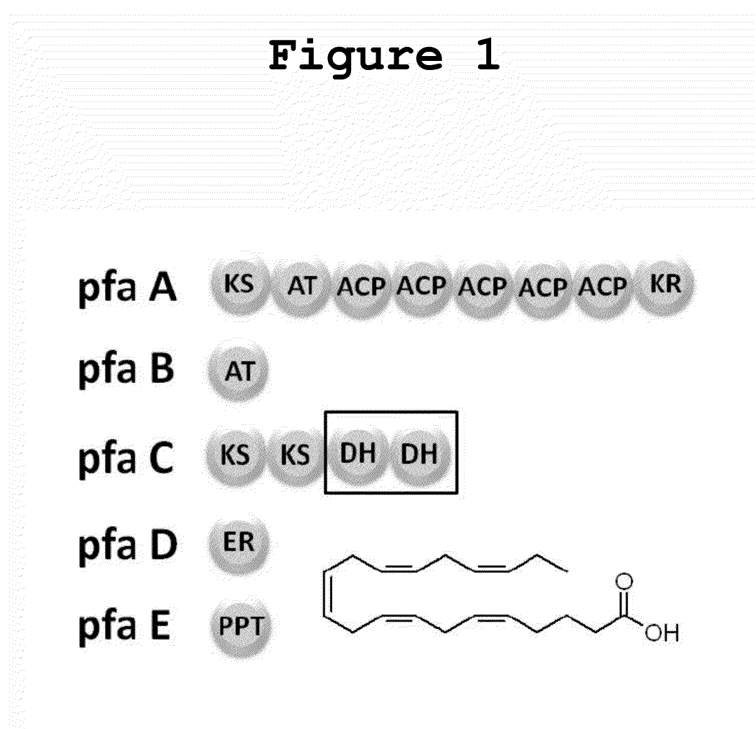

The pfaC protein of the PUFA synthase complex harbors two homologues of FabA/Z dehydratases as shown in FIG. 1.

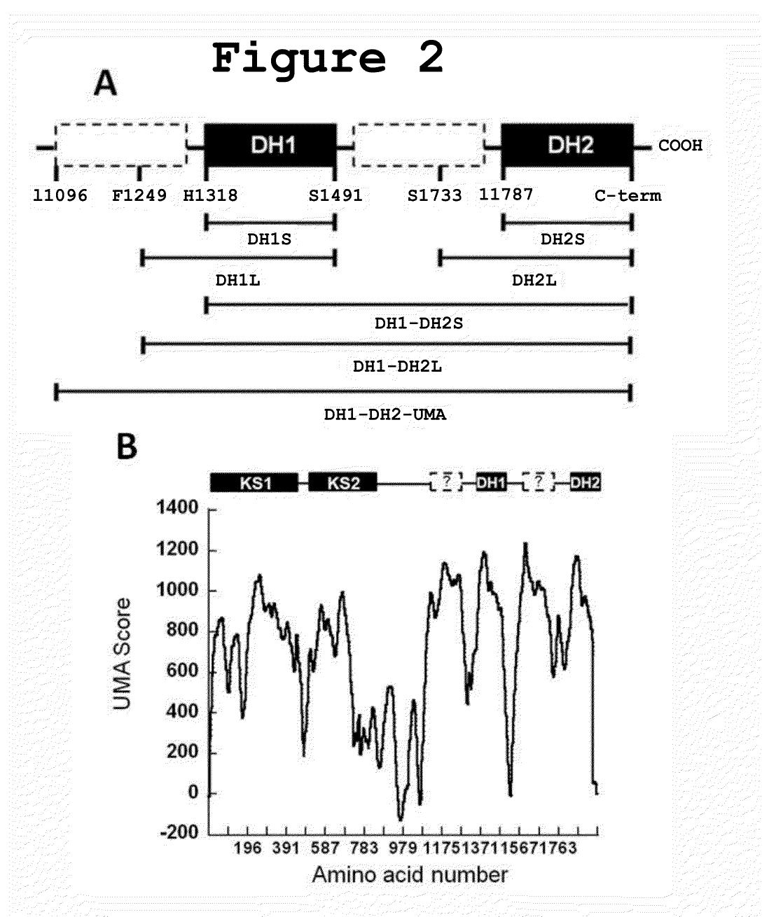

Initially, a number of protein constructs were designed on the basis of FabA homology and sequence conservation alone as summarized in FIG. 2A. Two “short” fragments, DH1S (H1318-S1491) and DH2S (I1787-C-term) were designed to include only the sequence homologous to FabA. Two “longer” fragments, DH1L (F1249-S1491) and DH2L (S1733-C-term), were designed to include additional conserved sequence N-terminal to the FabA-homology region. Finally, to explore the possibility that the two FabA-homology regions stabilized one another, we also generated protein fragments which contained both Fab-homology regions, DH1-DH2S (H1318-C-Term) and DH1-DH2L (F1249-C-Term). All of these protein fragments were expressed as GST fusion proteins and as His-tagged proteins in E coli and all were found to be insoluble as evidenced by their presence in the lysis pellet (data not shown).

In order to more accurately define the boundaries for the putative DH domains from pfaC so as to increase the likelihood of generating a functional enzyme fragment, we analyzed the sequence using the Udwary-Merski Algorithm (UMA) which assigns a numerical score to each amino acid based on the probability that it is located within a structured domain, as opposed to it being located in an unstructured linker region. UMA analysis of the pfaC sequence revealed six domain regions as defined by their high UMA score as shown in FIG. 2B. Four of the six domains had been previously identified based on sequence alignments: two KS domains in the N-terminal portion of the protein (KS1 and KS2), and the two FabA homologs (DH1 and DH2). The two other areas of high UMA score were located directly N-teminal to the putative DH domains. The predicted secondary structure for the two new pseudodomains (termed DH1′ and DH2′) was that of a hot-dog fold, much like the predicted secondary structure for the Fabhomology domains.

Based on the UMA analysis and on the secondary structure prediction, fragment DH1-DH2-UMA (I1096-N-Term) was designed and expressed as a His-tagged protein in soluble form. After nickel resin purification and anion exchange chromatography, a total yield of 1.0 mg of pure protein was obtained per liter of culture. Gel filtration chromatography of this protein revealed an equilibrium between a monomer and a dimer in equal proportions (data not shown).

Preliminary Activity of DH1-DH2-UMA.

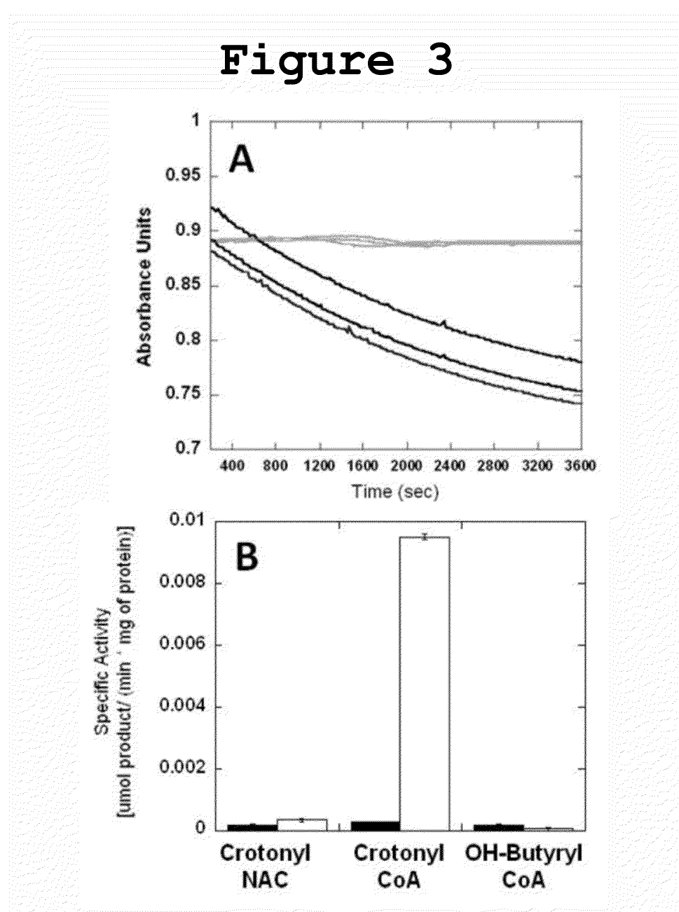

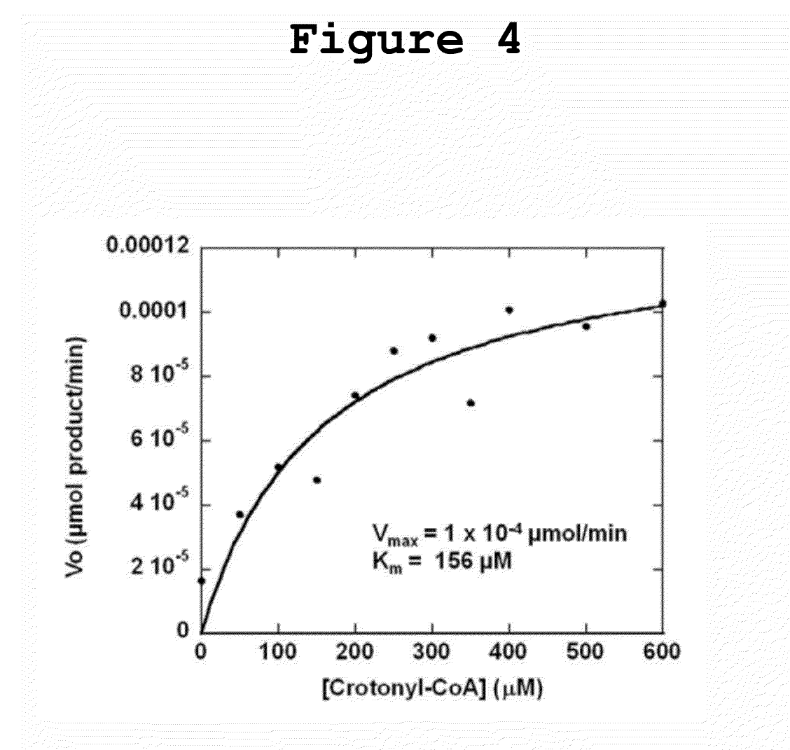

Incubation of DH1-DH2-UMA with crotonyl-CoA resulted in a decrease in the absorbance at 260 nm, consistent with the hydration of the double bond as shown in FIG. 3. The N-acetyl cysteamine (NAC) thioester of crotonic acid was not hydrated suggesting the importance of the pantetheine carrier for substrate recognition. The dehydration of the β-hydroxybutyryl-CoA was also monitored but no activity was detected in the forward reaction, probably due to the fact that the chemical equilibrium favors the reverse reaction. Initial efforts to measure the Michaelis-Menten kinetic parameters were frustrated by the fact that the amount of substrate required to saturate the enzyme was too high for spectrophotometric determination at a wavelength of 260 nm. In order to lower the absorption intensity of the acyl-CoA substrate, the reaction was monitored at a wavelength of 235 nm and the initial velocity was measured at different substrate concentration. From the saturation curve for crotonyl-CoA, the kinetic parameters were extracted (FIG. 4: Vmax=0.0001 μmol product/min; Km=156 μM). The measured activity towards NAC-loaded substrates or β-hydroxybutyryl-CoA was too low to yield a reliable saturation curve for the determination of the kinetic parameters.

Effect of DH1-DH2-UMA Overexpression on the Fatty Acid Profile of E coli.

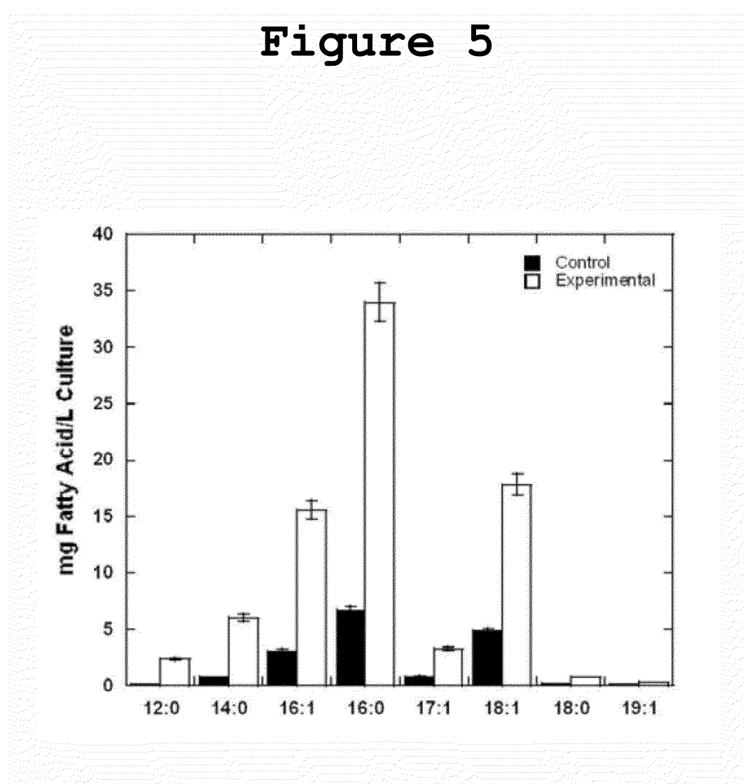

The overexpression of enzymes has been employed as a strategy to enhance fatty acid production or to alter the normal fatty acid profile of E coli. In order to investigate whether DH1-DH2-UMA would interact with the fatty acid biosynthesis machinery of E coli and result in the formation of polyunsaturated fatty acids, we measured the production of fatty acids in a strain overexpressing DH1-DH2-UMA. No polyunsaturated fatty acids were detected in any of the bacterial extracts, indicating that the expression of DH1-DH2-UMA is not sufficient to catalyze the formation of multiple cis double bonds in the fatty acids normally made by E coli. It was observed, however, a 4-fold to 5-fold increase in the total production of free saturated and monounsaturated fatty acids without a change in the percentage composition of fatty acids as shown in FIG. 5. The fact that the expression of DH1-DH2-UMA affected the production of all fatty acids in equal proportions suggests that the protein is capable of interacting with the E coli machinery for fatty acid biosynthesis in a way that does not discriminate based on fatty acid chain length.

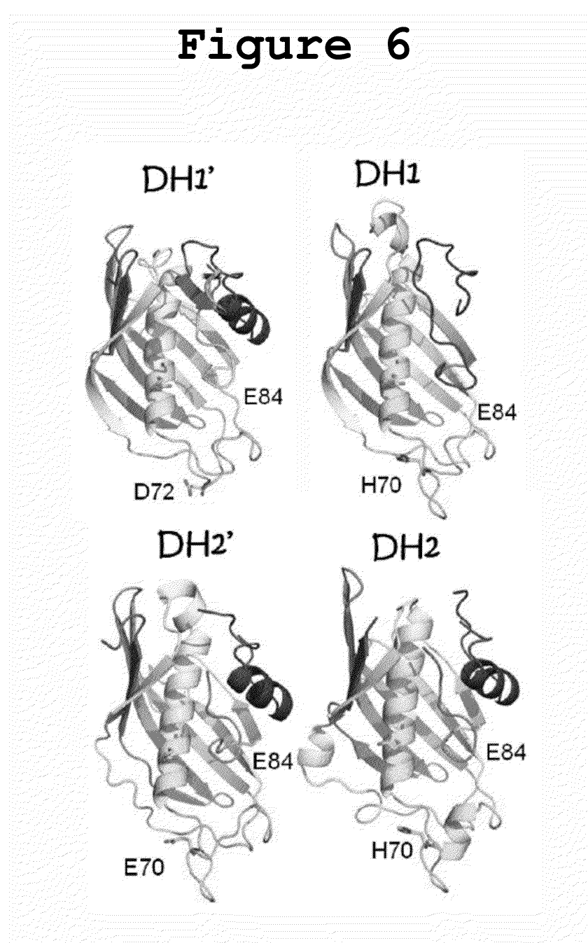

Three-dimensional models of DH domains and pseudodomains. In order to verify the presence of amino acid residues normally associated with dehydratase activity, we built three-dimensional models of all domains and pseudo-domains using the Phyre Server from Imperial College London as shown in FIG. 6. The 3D models generated for the actual Fab-homology domains (DH1 and DH2) feature an active site His70 and the conserved G1u84 typical of dehydratases (amino acid sequence numbers are based on the FabA numbering). Interestingly, even though the newly identified N-terminal pseudodomains (DH1′ and DH2′) do not have a high enough sequence similarity with any known protein, their secondary structure prediction in the Phyre server was found to be consistent with the formation of a hotdog fold, possibly the first half of a double hotdog. Instead of the expected His70 conserved in dehydratases, DH1′ featured an Asp72 and DH2′ featured a Glu70 in the corresponding region as shown in FIG. 6. These acidic residues in the active site are not typically observed in the hotdog dehydratases but they are a defining feature of the hotdog hydrolases, suggesting a possible involvement of DH1′ and DH2′ in hydrolysis.

Discussion

The biosynthesis of PUFAs in deep-sea bacteria is carried by a family of enzymes that contain a unique and conserved arrangement of enzyme domains. PUFA synthases have been found in metagenomic DNA from marine samples collected throughout the world, indicating that anaerobic PUFA biosynthesis is a widely selected mechanism for microbial adaptation to high-pressure and low temperature environments. Despite much interest in elucidating how the PUFA synthase carries out its function, published work on the enzymatic activities of PUFA synthases has been sparse. Bumpus et al., 2008 showed for the first time the in vitro activity of the enoyl reductase (pfaD) enzyme from Shewanella oneidensis PUFA synthase and Jiang et al., 2008 interrogated the role of the tandem ACP arrangement, which is a hallmark of PUFA synthases. The present invention addressed another conserved feature of PUFA synthases, a pair of conserved DH domains arranged in tandem near the C-terminus of the multidomain protein, pfaC.

Analysis of the sequence of pfaC protein using the Udwary-Merski Algorithm revealed the presence of two new pseudodomains located directly N-terminal to the regions of FabA homology. These pseudodomains were found to be essential for the proper expression of protein fragments, since only the protein fragments that included both pseudodomains were soluble, stable and active. This result alone would suggest that DH′ pseudodomains are important components of the three-dimensional structure of dehydratase domains. This finding also confirms the general applicability of the Udwary-Merski Algorithm for the identification of functional units within multidomain proteins with unknown functions or from unexplored lineages.

The predicted secondary structure for both DH′ pseudodomains was that of a hotdog fold, which is also the expected three-dimensional topology of the FabA-homology DH domains. This predicted arrangement of contiguous hotdog folds points towards an overall double hotdog structure, which has become the widely accepted model for embedded dehydratases based on structural and biochemical evidence. However, several differences exist between the PUFA arrangement and its FAS and PKS evolutionary cousins. While in FAS/PKS DH, the pseudodomains are located C-terminal to the Fabhomology domain, in the PUFA DH, the pseudodomains are located N-terminal to the Fab-homology domain. This alternative gene structure of the PUFA DH suggests a tandem gene duplication event that took place independently in terrestrial FAS/PKS and marine PUFA synthase for the generation of functional DH dimers, resulting in two alternative convergent topological solutions. Another difference between FAS/PKS DH and PUFA DH is that, while FAS/PKS DH domains consist of didomains (one FabA homology domain plus one pseudodomain), the PUFA DH complex invariably consists of a tetradomain (two FabA homology plus two pseudodomains). This invention does not address the question of how the four protein domains are paired in the functional assembly. Additional structural characterization of DH1-DH2-UMA will have to be carried out in order to elucidate how the different domains are arranged in a functional complex.

Substantial work has been dedicated to determining the specific role of pseudodomains in the activity of FAS DH domains beyond stabilizing the dimeric structure by partnering with the FabA-homology domain. Amino acids in the DH pseudodomain have been implicated in the partial activity of the FAS ketoreductase domain. Additionally, an Asp residue in the FAS pseudodomain has been found to be essential and a Gln residue in the pseudodomain has been found to be important for dehydratase activity. In the PUFA DH in this report, multiple sequence alignment of the pseudodomains reveal levels of sequence conservation (67% and 71% for DH1′ and DH2′, respectively) that were comparable to the sequence conservation of the FabA homolgy domains (61% and 75% for DH1 and DH2, respectively). This high level of sequence similarity among the pseudodomains is suggestive of a role in DH function beyond that of a structural scaffold for dimerization.

The soluble DH1-DH2-UMA fragment was competent to catalyze the hydration of crotonyl-CoA with a specific activity of 0.009 μmol product/(min*mg enzyme). When this number is converted to the units of specific activity employed in Pasta et al., 2007, it becomes 0.83 mol product/(min*mol enzyme), at least two orders of magnitude lower than the specific activity reported for the FAS 1-1168 construct (204 mol product/(min*mol enzyme)). It has been shown that dehydratase activity decreases dramatically with decreasing length of the acyl chain. Although that report does not include the activity toward crotonyl-ACP (3:1), the difference between the specific activity against octenoyl-ACP (8:1) and butenoyl-ACP (4:1) was about one order of magnitude. In addition a similar dramatic effect was observed when comparing ACP-linked substrate to pantetheine-linked substrates. The PUFA DH in this report was assayed for activity against crotonyl-CoA (3:1). Thus, it is not surprising that the specific activity is low considering that the acyl chain is even shorter that the shortest one in Pasta et al., 2007 and that the substrate in this report is not loaded on an ACP. Further work will need to be carried out to determine the substrate preference for PUFA DH domains in a more physiological context.

Additional confirmation of the activity of DH1-DH2-UMA came from measuring the effect of its overexpression on the production of fatty acids in E coli. According to the invention, a significant increase in the production of fatty acids was observed in the BL21 E coli strain expressing the DH1-DH2-UMA protein. Previous work by others has shown that overexpression of the E coli FabA dehydratase does not increase the production of fatty acids in E coli. Thus, it is hard to argue that the observed increase in fatty acid production in this report is due to the dehydratase activity of DH1-DH2-UMA although it cannot be entirely ruled out. It has been well established that the overexpression of thioesterases and other hydrolases results in the enhancement of the production of fatty acids and other high-energy biofuel precursors. Therefore, it is possible that an adventitious or unphysiological hydrolase activity, possibly an artifact arising from high enzyme concentration inside overexpressing bacterial cells, could be responsible for the observed enhancement of fatty acid production in E coli.

Inspection of the homology model made for the DH′ pseudodomains reveals a hotdog fold similar to that expected for the FabA-homology regions, although with a different amino acid occupying the active site position as shown in FIG. 6. While the model for the FabA-homology region contains a His residue in position 70 and a Glu in position 84, consistent with dehydratase function, the homology model for the DH′ pseudodomains reveals a Glu70 and a Glu84, which are more commonly found in hotdog hydrolases than in dehydratases. There have been reports of bona fide DH domains with the His70 and Glu84, that have hydrolase activity. Moriguchi et al. 2010 reported a hidden thioesterase function in what appeared by sequence homology to be an embedded dehydratase domain in the 6-MSA Synthase fungal multienzyme. The thioesterase activity in that domain was abolished when the conserved active site His70 residue was replaced by Ala, thus showing that an apparent DH domain could catalyze either dehydration or hydrolysis.

Therefore, based on our results and on the three-dimensional models for the DH domains according to the invention, it cannot be ruled out that the DH tetradomain of PUFA synthases houses a hydrolase or esterase activity in addition to the reported dehydratase activity.

Although the present invention has been described herein with reference to the foregoing exemplary embodiment, this embodiment does not serve to limit the scope of the present invention. Accordingly, those skilled in the art to which the present invention pertains will appreciate that various modifications are possible, without departing from the technical spirit of the present invention.

We claim:

1. A modified Escherichia coli (E. coli) comprising:
E. coli having genetically inserted an over-expressed dehydratase gene from Photobacterium profundum.

2. The modified E. coli of claim 1, wherein said dehydratase gene comprises a DH1-DH2 protein, the boundaries of said protein being determine by the Udwary-Merski Algorithm (UMA).

3. The modified E. coli of claim 1, wherein said modified E. coli produces about 5-10 times more free fatty acids than wild-type E. coli.

4. The modified E. coli of claim 1, wherein said dehydratase gene is over-expressed at about room temperature.

5. A method of increasing the production of free fatty acids on E. coli comprising:
genetically modifying E. coli by inserting an over-expressed dehydratase gene from Photobacterium profundum into E. coli.

6. The method of claim 5, wherein said dehydratase gene is over-expressed at about room temperature.

7. The method of claim 5, wherein said dehydratase gene comprises a DH1-DH2 protein, the boundaries of said protein being determine by the Udwary-Merski Algorithm (UMA).


SEQUENCE LISTING (17 sequences)

SEQ ID NO: 1 - DNA, 38 bp, Artificial Sequence
  Sequence source PCR primer, 38 bases
catgcatgggatccaacttgctagacgcaaatatcgca

SEQ ID NO: 2 - DNA, 39 bp, Artificial Sequence
  Sequence source PCR primer, 39 bases
catgcatgcccgggtcatgattcttctttgatcatcacg

SEQ ID NO: 3 - DNA, 28 bp, Artificial Sequence
  Sequence source PCR primer, 28 bases
caccttctcttacgaatgtttcgttggc

SEQ ID NO: 4 - DNA, 39 bp, Artificial Sequence; identical to SEQ ID NO: 2
  Sequence source PCR primer, 39 bases

SEQ ID NO: 5 - DNA, 39 bp, Artificial Sequence
  Sequence source PCR primer, 39 bases
catgcatgggatccaacttactggataaagaaagccgtt

SEQ ID NO: 6 - DNA, 24 bp, Artificial Sequence
  Sequence source PCR primer, 24 bases
tcaggcttcttcaatacagattgc

SEQ ID NO: 7 - DNA, 37 bp, Artificial Sequence
  Sequence source PCR primer, 37 bases
catgcatgggatccttcagcttcgaactcagtaccga

SEQ ID NO: 8 - DNA, 24 bp, Artificial Sequence
  Sequence source PCR primer, 24 bases
tcaggcttcttcaatacagattgc

SEQ ID NO: 9 - DNA, 28 bp, Artificial Sequence
  Sequence source PCR primer, 28 bases
caccaacttgctagacgcaaatatcgca

SEQ ID NO: 10 - DNA, 24 bp, Artificial Sequence
  Sequence source PCR primer, 24 bases
tcaggcttcttcaatacagattgc

SEQ ID NO: 11 - DNA, 28 bp, Artificial Sequence
  Sequence source PCR primer, 28 bases
caccttctcttacgaatgtttcgttggc

SEQ ID NO: 12 - DNA, 24 bp, Artificial Sequence
  Sequence source PCR primer, 24 bases
tcaggcttcttcaatacagattgc

SEQ ID NO: 13 - DNA, 27 bp, Artificial Sequence
  Sequence source PCR primer, 27 bases
cacccgcaaaccttgtatctgggatta

SEQ ID NO: 14 - DNA, 24 bp, Artificial Sequence
  Sequence source PCR primer, 24 bases
tcaggcttcttcaatacagattgc

SEQ ID NO: 15 - DNA, 5741 bp, Artificial Sequence
  Plasmid vector; pET200TOPO
caaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgcggggttctcatcatcatcatcatcatggtatggctagcatgactggtggacagcaaatgggtcgggatctgtacgacgatgacgataaggatcatcccttcaccaagggcgagctcaacgatccggctgctaacaaagcccgaaaggaagctgagttggctgctgccaccgctgagcaataactagcataaccccttggggcctctaaacgggtcttgaggagttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaactgacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgggcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatg

SEQ ID NO: 16 - DNA, 14994 bp, Escherichia coli
aaattgaagagtttgatcatggctcagattgaacgctggcggcaggcctaacacatgcaagtcgaacggtaacaggaaacagcttgctgtttcgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggatgaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctccttaccttaaagaagcgtactttgcagtgctcacacagattgtctgatgaaaatgagcagtaaaacctctacaggcttgtagctcaggtggttagagcgcacccctgataagggtgaggtcggtggttcaagtccactcaggcctaccaaatttgcacggcaaatttgaagaggttttaactacatgttatggggctatagctcagctgggagagcgcctgctttgcacgcaggaggtctgcggttcgatcccgcatagctccaccatctctgtagtgattaaataaaaaatacttcagagtgtacctgcaaaggttcactgcgaagttttgctctttaaaaatctggatcaagctgaaaattgaaacactgaacaacgaaagttgctcgtgagtctctcaaattttcgcaactctgaagtgaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatcaaattgaagagtttgatcatggctcagattgaacgctggcggcaggcctaacacatgcaagtcgaacggtaacaggaagcagcttgctgcttcgctgacgagtggcggacgggtgagtaatgtctgggaaactgcctgatggagggggataactactggaaacggtagctaataccgcataacgtcgcaagaccaaagagggggaccttagggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctccttaccttaaagaagcgtactttgcagtgctcacacagattgtctgatagaaagtgaaaagcaaggcgtcttgcgaagcagactgatacgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaatgaaagttgttcgtgagtctctcaaattttcgcaactctgaagtgaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaaaattgaagagtttgatcatggctcagattgaacgctggcggcaggcctaacacatgcaagtcgaacggtaacaggaagcagcttgctgcttcgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctccttaccttaaagaagcgtactttgcagtgctcacacagattgtctgatgaaaatgagcagtaaaacctctacaggcttgtagctcaggtggttagagcgcacccctgataagggtgaggtcggtggttcaagtccactcaggcctaccaaatttgcacggcaaatttgaagaggttttaactacatgttatggggctatagctcagctgggagagcgcctgctttgcacgcaggaggtctgcggttcgatcccgcatagctccaccatctctgtagtgattaaataaaaaatacttcagagtgtacctgcaaaggttcactgcgaagttttgctctttaaaaatctggatcaagctgaaaattgaaacactgaacaacgaaagttgttcgtgagtctctcaaattttcgcaacacgatgatgaatcgcaagaaacatcttcgggttgtgaggttaagcgactaagcgtacacggtggatgccctggcagtcagaggcgatgaaggacgtgctaatctgcgataagcgtcggtgaggtgatatgaaccgttataaccggcgatttccgaatggggaaacccagtgtgattcgtcacactatcattaaattgaagagtttgatcatggctcagattgaacgctggcggcaggcctaacacatgcaagtcgaacggtaacaggaagcagcttgctgcttcgctgacgagtggcggacgggtgagtaatgtctgggaaactgcctgatggagggggataactactggaaacggtagctaataccgcataacgtcgcaagaccaaagagggggaccttagggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctccttaccttaaagaagcgtactttgcagtgctcacacagattgtctgatagaaagtgaaaagcaaggcgtcttgcgaagcagactgatacgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaatgaaagttgttcgtgagtctctcaaattttcgcaactctgaagtgaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaaaattgaagagtttgatcatggctcagattgaacgctggcggcaggcctaacacatgcaagtcgaacggtaacaggaaacagcttgctgtttcgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctccttaccttaaagaagcgtactttgcagtgctcacacagattgtctgatgaaaatgagcagtaaaacctctacaggcttgtagctcaggtggttagagcgcacccctgataagggtgaggtcggtggttcaagtccactcaggcctaccaaatttgcaccgcaaatttgaagaggttttaactacatgttatggggctatagctcagctgggagagcgcctgctttgcacgcaggaggtctgcggttcgatcccgcatagctccaccatctctgtagtgattaaataaaaaatacttcagagtgtacctgcaaaggttcactgcgaagttttgctctttaaaaatctggatcaagctgaaaattgaaacactgaacaacgaaagttgttcgtgagtctctcaaattttcgcaacacgatgatgaatcgcaagaaacatcttcgggttgtgaggttaagcgactaagcgtacacggtggatgccctggcagtcagaggcgatgaaggacgtgctaatctgcgataagcgtcggtgaggtgatatgaaccgttataaccggcgatttccgaatggggaaacccagtgtgattcgtcacactatcattaaattgaagagtttgatcatggctcagattgaacgctggcggcaggcctaacacatgcaagtcgaacggtaacaggaaacagcttgctgtttcgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctccttaccttaaagaagcgtactttgcagtgctcacacagattgtctgataaaaagtgaaaagcaaggcgtcttgcgaagcagactgatacgtccccttcgtctagaggcccaggacaccgccctttcacggcggtaacaggggttcgaatcccctaggggacgccacttgctggtttgtgagtgaaagtcacctgccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcaaattgaagagtttgatcatggctcagattgaacgctggcggcaggcctaacacatgcaagtcgaacggtaacaggaagcagcttgctgcttcgctgacgagtggcggacgggtgagtaatgtctgggaaactgcctgatggagggggataactactggaaacggtagctaataccgcataacgtcgcaagaccaaagagggggaccttagggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctccttaccttaaagaagcgtactttgcagtgctcacacagattgtctgatagaaagtgaaaagcaaggcgtcttgcgaagcagactgatacgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaatgaaagttgttcgtgagtctctcaaattttcgcaactctgaagtgaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcga

SEQ ID NO: 17 - DNA, 1755 bp, Photobacterium profundum
tcatgatgcctttccctctttttgcgcccatacagccagtttttctctaaaaatcttggcgttatggcgtatatcaacgggaaaattagagtgaattagaaaatcgacaatacccacagtttgttgatgtttagcgccaatcgttttaagctcggcaaaaaacgtattacgggcagtatcatccgtcaggcttgtgcctttattaagctcaacacacagcagtggcactgtttcaccatgacgttcaatgccgaccaatgcggtacgcttgaccgaagagtgagtattaaatatacgttcacaaggaatagaaaaatacggcagcttgtctgatgcaaccgcaacgcgattggcttcaacacggtgagctttacgaccacacatccacagttgcccgctgtcgtccaagtagcctaaatcccccatgcggtgacgtacagtattcgccccatcggtaattttagcctgaacggttgcatgatcacggtgataatacgcacggctcaccatgggaccttttacaacgatttcgccaatttggtttacaggtaaacgtaacgtttcgtcccacgtagtaattggatcgtcggtgatcgcaatgatcgcaatatccacaccatcaacagcttgaccgacacaaataccaccgccattatcagtgacatccgtggttttcattaactgatcactaccaatcatggtaagaggcaatgattctgtcgcaccataagaattcagtacttcaaccccatcctttagcattttactaaagcgtgaaatagacgaaatagtagcaggtgcgccagcggaaataacacgtttgatactgggtaatgtatgaggtttatgatgctgtgtacccgctttccctaaacgctctatcaacgcaggattcacaaacatattggtacattggtattgttcaatcgctgcaaacaaactgtcgggattagccgttattggtttactggcatccatatcaggcacaatcgatgccatgcctaaagctggaccaaaaagagagaataacgggaaggtcgcaagatcacgctcgccgtgggcgataccataatcatttttcaatacactgatttgtgcttcaaacattgcatgggtatacacaacacctttcggcgtacccgtactgccactggtaaatagaatggctgccatttcatcatttttaagcttggcaatatcataatcaatcgcttgatgcattttactgcgcggcttaataacagtattacgtttaagtaatgcttttagtgttgtaccaccaaaaatattggcaaatacatcatttccaccaacggtgagtaaacgtcgaacactcggtttaccccaaccaaataaacaacgggcaatatgcgctttagggataccaataaacgcatcgggcttagcttcatcaaagcactgtttgaggtttttaacccccatgccaggatcaaccaaaataggaacaatacccgctttaaataatgcaaatgttagagcaaaaaaatcaagactaggcgttaccatcagtaccgctttcatgcctcgtttaataccgtgatcattcaacgcttgtgcaattgcgttactgtcagtgtttaactgcccaaacgttaattcttcataccgtagtttgccgaataacgatcgtttttgaacagcaacagcaagcgaagcaggtgtttcttttgccgcgcgtgttaagtggcgacagatattggctttagaggcatcaatctcatgccctttatccat